(19) United States
(12) Patent Application Publication
(10) Pub. No.: US 2012/0330061 A1
(43) Pub. Date: Dec. 27, 2012

(54) PREPARATION METHOD OF N,N'-DIALKYL-3,3'-DITHIODIPROPIONAMIDE

(75) Inventors: Jae-Min Ha, Gyeonggi-do (KR); Jeong-Soo Yu, Gyeonggi-do (KR); Tae-Woong Lee, Gyeonggi-do (KR); Jeong-Joo Shin, Gyeonggi-do (KR)

(73) Assignee: SK Chemicals Co., Ltd., Kyungki-do (KR)

(21) Appl. No.: 13/515,990

(22) PCT Filed: Dec. 6, 2010
(86) PCT No.: PCT/KR2010/008676

§ 371 Date: Sep. 10, 2012

(30) Foreign Application Priority Data
Dec. 16, 2009 (KR) 10-2009-0125452

(51) Int. Cl. C07C 231/02

(52) U.S. Cl. 564/136

(57) ABSTRACT

This disclosure relates to a method for preparing N,N′-dimethyl-3,3′-dithiodipropionamides, which is an intermediate compound used for preparation of substituted 3-isothiazolones, including reacting 3,3′-dithiopropionic acid alkyl ester with an alkylamine at a temperature of from 0 to 50° C. in the presence of a polar solvent.

### FIELD OF THE INVENTION

The present invention relates to a method for preparing N,N′-dimethyl-3,3′-dithiodipropionamides, which is an intermediate compound used for preparation of substituted 3-isothiazolones.

### BACKGROUND OF THE INVENTION

Currently, 3-isothiazolones are generally used as bactericide or antibacterial and antimicrobial agent added to paint, cosmetics, fiber, plastics, and the like.

As a method for preparing 3-isothiazolones, U.S. Pat. No. 4,868,310 discloses a method of preparing N-substituted-3-mercaptopropionamide by simultaneously supplying a mixture of unsaturated nitrile and alcohol and a strong inorganic acid in an appropriate organic solvent to form acrylamide, and treating it with a thiolating agent.

U.S. Pat. No. 4,052,440 describes a method of reacting acrylic acid and hydrogen sulfide in the presence of a weak base amine catalyst to produce mercaptopropionic acid methyl ester or polythiodimethyldipropionester, and then obtaining 3,3′-dithiopropionic acid methyl ester

U.S. Pat. No. 4,067,901 suggests a method of continuously separating dithiodimethyldipropionester by reacting methyl acrylate and hydrogen sulfide using polythiodimethyldipropionester as an active reaction solvent.

U.S. Pat. Nos. 4,939,266 and 5,068,338 suggest a preparation method using methylmercaptopropionate as a starting material, wherein the product does not contain N-methyl-3-(N-methylamino)-propionamide (MMAP). However, this method is economically infeasible due to high cost of starting materials, and is dangerous because of a high handling risk

U.S. Pat. Nos. 5,312,827 and 5,420,290 describe a preparation method using 3,3′-dithiopropionic acid methyl ester (DDD) as a starting material. In this case, N,N′-dimethyl-3,3′-dithiodipropionamide (DDDA) prepared by the method contains an N-methyl-3-(N-methylamino)-propionamide (MMAP) impurity at a high concentration of 0.5% to 1.1%. To remove the impurity, methods of recrystallizing N,N′-dimethyl-3,3′-dithiodipropionamide (DDDA) containing the impurity in an alcohol organic solvent, extracting with an organic solvent, and decreasing nitrosamine and a precursor thereof using an ion exchange resin are disclosed. However, these methods fail to suggest a fundamental method of inhibiting production of N-methyl-3-(N-methylamino)-propionamide (MMAP), which is an impurity generated in the synthesis reaction of 3,3′-dithiodimethyldipropionic acid ester (DDD) and an amine, and further purification should be conducted using a cation exchange resin in order to remove N-methyl-3-(N-methylamino)-propionamide (MMAP), and thus these methods are economically unfavorable.

### DETAILED DESCRIPTION OF THE INVENTION

### Technical Objectives

To overcome the above problems, during repeated studies on a method for preparing highly pure N,N′-dialkyl-3,3′-dithiodipropionamides that include little N-methyl-3(N-methylamino)-propionamide (MMAP), the inventors completed the present invention.

### Technical Solution

In order to achieve the object, one aspect of the invention provides a method for preparing N,N′-dialkyl-3,3′-dithiopropionamide including: reacting 3,3′-dithiopropionic acid alkyl ester of the following Chemical Formula 2 with an alkylamine of the following Chemical Formula 3 at a temperature of from 0 to 50° C. in the presence of a polar solvent; solidifying N,N′-dialkyl-3,3′-dithiodipropionamide of the following Chemical Formula 1 included in the reaction solution; and drying the solid material of N,N′-dialkyl-3,3′-dithiodipropionamide.

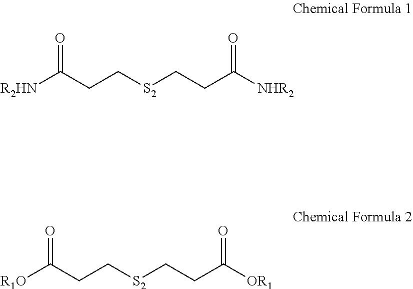

R₂NH₂ Chemical Formula 3

In Chemical Formulae 1 to 3, R₁ and R₂ are independently hydrogen or a C1-2 alkyl group.

According to one embodiment, the polar solvent may be an alcohol including a C1-10 linear or branched alkyl group, water, or a combination thereof.

According to one embodiment, the water may be introduced in the content of 100 to 700 parts by weight, based on 100 parts by weight of the 3,3′-dithiopropionic acid alkyl ester.

According to one embodiment, the reaction may be progressed while further adding a reducing agent of an aqueous thiosulfite-based inorganic salt or a sulfite-based inorganic salt.

According to one embodiment, the inorganic salt reducing agent may be added in the equivalent of 0.1 to 1.0, based on 1 equivalent of an alkylamine.

According to one embodiment, the reducing agent may be selected from Na₂S₂O₃, Na₂SO₃, K₂S₂O₃, K₂SO₃, and a combination thereof.

According to one embodiment, the solidification of the N,N′-dialkyl-3,3′-dithiodipropionamide may be progressed at 0 to 30° C. for 1 to 3 hours.

According to one embodiment, the solidification may be progressed while further adding at least one inorganic salt selected from the group consisting of sodium sulfate, ammonium sulfate, sodium chloride, ammonium chloride, magnesium sulfate, and magnesium chloride.

According to one embodiment, if the inorganic is added, the inorganic salt may be introduced in the content of 1 to 10 parts by weight, based on 100 parts by weight of the reaction solution supplied in the solidification step.

By adding the inorganic salt in the solidification step, solubility of N,N′-dialkyl-3,3′-dithiodipropionamide in the reaction solution may be maintained at 5% or less.

According to one embodiment, the solidified N,N′-dialkyl-3,3′-dithiodipropionamide may be separated from the reaction solution by centrifugal filtration.

According to one embodiment, the centrifugal filtration may be progressed such that moisture content of the solidified N,N′-dialkyl-3,3′-dithiodipropionamide may be 20 wt % or less.

According to one embodiment, the reaction temperature may be 0 to 25° C.

According to one embodiment, drying may be progressed such that moisture content of the N,N′-dialkyl-3,3′-dithiodipropionamide may become 0.1 wt % or less.

Hereinafter, the present invention will be explained in detail.

During repeated studies on a method for preparing highly pure N,N′-dialkyl-3,3′-dithiodipropionamides that include little N-methyl-3(N-methylamino)-propionamide (MMAP), the inventors completed the present invention.

Specifically, it was found out that if a reaction is conducted under specific conditions in the presence of a polar solvent, substantially no N-methyl-3-(N-methylamino)-propionamide (MMAP) remains in N,N′-dialkyl-3,3′-dithiodipropionamide prepared from 3,3′-dithiodipropionic acid alkyl ester and an alkylamine, and the present invention was completed.

Meanwhile, it was confirmed that the result is due to inhibition of formation of N-methylacrylamide, which is temporarily produced at the beginning of formation of N-methyl-3-(N-methylamino)-propionamide (MMAP). That is, it was found that if a polar solvent is used as a reaction solvent, and the reaction is conducted at 0 to 50, formations of N-methylacrylamide and N-methyl-3-(N-methylamino)-propionamide (MMAP), known carcinogens, are inhibited, and the present invention was completed.

Specifically, the inventors found that if preparation of N,N′-dialkyl-3,3′-dithiodipropionamide by the reaction of 3,3′-dithiodipropionate dialkyl ester and an alkylamine is progressed in the presence of a polar solvent, little N-methyl-3-(N-methylamino)-propionamide (MMAP) is detected in the produced N,N′-dialkyl-3,3′-dithiodipropionamide, and the reaction object N,N′-dialkyl-3,3′-dithiodipropionamide may be prepared with high purity.

A method for preparing N,N′-dialkyl-3,3′-dithiodipropionamide according to one embodiment includes:

reacting 3,3′-dithiopropionic acid alkyl ester of the following Chemical Formula 2 with an alkylamine of the following Chemical Formula 3 at 0 to 50° C. in the presence of a polar solvent;

solidifying N,N′-dialkyl-3,3′-dithiodipropionamide of the following Chemical Formula 1 included in the reaction solution; and

drying the solid material of N,N′-dialkyl-3,3′-dithiodipropionamide.

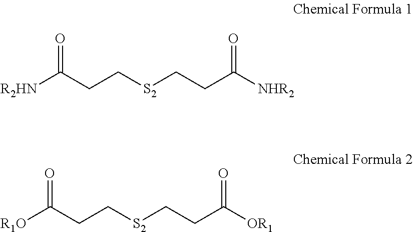

R₂NH₂ Chemical Formula 3

In Chemical Formulae 1 to 3, R₁ and R₂ are independently hydrogen or a C1-2 alkyl group.

One embodiment of the reaction steps is shown in the following Reaction Equation 1.

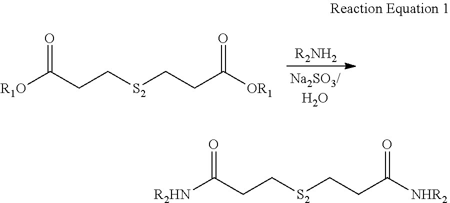

In Reaction Equation 1, R₁ and R₂ are independently hydrogen or a C1-2 alkyl group.

Furthermore, as shown in Reaction Equation 1, if 3,3′-dithiodipropionic acid alkyl ester and alkylamine (R₂NH₂) are reacted in the presence of a polar solvent while further adding a reducing agent of a thiosulfite-based inorganic salt or a sulfite-based inorganic salt, N,N′-dialkyl-3,3′-dithiodipropionamide may be obtained with high purity.

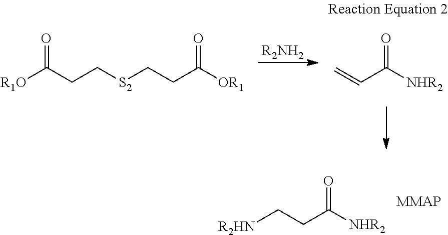

In Reaction Equation 2, R₁ and R₂ are independently hydrogen or a C1-2 alkyl group.

For reference, Reaction Equation 2 schematically shows formation of N-methyl-3-(N-methylamino)-propionamide (MMAP) in the preparation of N,N′-dialkyl-3,3′-dithiodipropionamide by the reaction of 3,3′-dithiodipropionic acid alkyl ester and alkylamine.

According to the present invention, formation of N-methyl-3-(N-methylamino)-propionamide (MMAP) as shown in Reaction Equation 2 may be inhibited.

The polar solvent may include at least one selected from the group consisting of an alcohol including a C1-10 linear or branched alkyl group, and water, but is not limited thereto. Examples of the alcohol including a C1-10 linear or branched alkyl group may include methanol, ethanol, propanol, isopropanol, butanol, and the like, and the alcohol may be used to improve yield of N,N′-dialkyl-3,3′-dithiodipropionamide and minimize contents of impurities such as MMAP.

More preferably, the polar solvent may be water, and the inventors confirmed that if the reaction is progressed in the presence of water, production of MMAP may be reduced to a detection limit.

Meanwhile, the polar solvent may be added in the content of 100 to 700 parts by weight based on 100 parts by weight of the 3,3′-dithiopropionic acid alkyl ester of Chemical Formula 2 in order to improve the yield of N,N′-dialkyl-3,3′-dithiodipropionamide and minimize impurities such as MMAP, but is not limited thereto. More preferably, it may be added in the content of 200 to 600 parts by weight based on 100 parts by weight of the 3,3′-dithiopropionic acid alkyl ester.

Further, the water may be added in the content of 100 to 700 parts by weight, more preferably 200 to 600 parts by weight, based on 100 parts by weight of the 3,3′-dithiopropionic acid alkyl ester of Chemical Formula 2.

In general, during the reaction of 3,3′-dithiodipropionic acid alkyl ester and alkylamine, hydrolysis of ester was expected to occur, and thus a polar solvent such as an aqueous solution solvent was hardly used.

However, the inventors found out that if 3,3′-dithiodipropionic acid alkyl ester and alkylamine are reacted in the presence of a polar solvent, phase separation occurs in the reaction solution and hydrolysis is inhibited by the phase separation, and thus production of N-methylacrylamide, which is a side-product generated when 3,3′-dithiodipropionic acid alkyl ester and alkylamine rapidly react, is extremely inhibited. Thus, it could be seen that N-methyl-3-(N-methylamino)-propionamide (MMAP), which is an impurity generated by chain reaction of alkylamine and N-methylacrylamide, is hardly produced.

If an organic solvent is used as the reaction solvent instead of the polar solvent, the phase separation does not occur. Thus, since the reaction is progressed in a homogeneous system without phase separation, the side reaction seriously occurs, and thus the production amount of N-methyl-3-(N-methylamino)-propionamide (MMAP) increases by several tens to several hundreds of times, compared to the reaction in the presence of a polar solvent.

Meanwhile, the reaction may be progressed while further adding a reducing agent such an aqueous thiosulfite-based inorganic salt or a sulfite-based inorganic salt. The reducing agent may be an inorganic reducing agent such as an alkali metal sulfite salt, a thioalkali metal sulfite salt, and the like.

Specifically, examples of the inorganic reducing agent may include Na₂S₂O₃, Na₂SO₃, K₂S₂O₃, K₂SO₃, and a combination thereof. If the reducing agent is added, yield of N,N′-dialkyl-3,3′-dithiodipropionamide may be increased, and remaining N-methyl-3-(N-methylamino)-propionamide (MMAP) in the product may be minimized. For this, Na₂SO₃ may be most preferably used.

For reference, the reaction rate of conversion into a sulfhydryl (SH—) compound by the reaction of the alkali metal sulfite salt or thioalkali metal sulfite salt and a polysulfide (Sn, n>2) compound is in the order of S₆²⁻>S₅²⁻>S₄²⁻>S₃²⁻>S₂²⁻, and it is known that the reaction rate becomes higher as the number of S becomes larger.

Meanwhile, the reducing agent such as the alkali metal sulfite salt or thioalkali metal sulfite salt may function for further inhibiting production of N-methylacrylamide, which is generated when the 3,3′-dithiodipropionic acid alkyl ester and alkylamine react. Thus, production of N-methyl-3-(N-methylamino)-propionamide (MMAP) may also be inhibited.

When sulfides including 3,3′-dithiodipropionic acid alkyl ester react with the alkali metal sulfite salt or thioalkali metal sulfite salt, an oxidation-reduction reaction of the sulfides occurs, and conversion of 3,3′-dithiodipropionic acid alkyl ester to N-methylacrylamide is inhibited under a reducing condition in the polar solvent. That is, the inventors confirmed that a sulfhydryl (RSH—) compound, which is produced by the reduction reaction of 3,3′-dithiodipropionic acid alkyl ester, reacts with N-methylacrylamide to inhibit production of N-methyl-3-(N-methylamino)-propionamide (MMAP), and completed the present invention.

The alkali metal sulfite inorganic salt or thioalkali metal sulfite inorganic salt may be preferably added in the content of 0.1 to 1.0 equivalents, based on 1 equivalent of the alkylamine. If the content is less than 0.1 equivalents, the effect of inhibiting production of N-methyl-3-(N-methylamino)-propionic acid (MMAP) according to the addition of the reducing agent may be insignificant, and if the content is greater than 1.0 equivalent, the increase in the inhibition effect of MMAP production according to the introduction amount may be insignificant, and thus it may be economically infeasible.

The reaction may be progressed at 0 to 50° C., and in order to more efficiently inhibit formation of side-product N-methylacrylamide, it may be preferably progressed at 0 to 25° C., more preferably 0 to 5° C.

Meanwhile, at a temperature of less than 0° C., when a polar solvent such as water is used, a reaction did not progress due to freezing, and at a temperature of greater than 50° C., the content of N-methyl-3-(N-methylamino)-propionamide (MMAP) increased 5 to 10% in the reaction solution.

According to another embodiment of the invention, there is provided a method of recovering highly pure N,N′-dialkyl-3,3′-dithiodipropionamide that does not contain N-methyl-3-(N-methylamino)-propionamide (MMAP), including solidifying N,N′-dialkyl-3,3′-dithiodipropionamide while controlling particle size of the N,N′-dialkyl-3,3′-dithiodipropionamide crystal.

The solidification may be preferably progressed such that the solidified crystal may have particle diameter of 100 μm to 2 mm. If the particle size is within the above range, impurities may be excluded by various physical techniques such as filtration and centrifugation, and a highly pure product may be obtained. When N,N′-dialkyl-3,3′-dithiodipropionamide is crystallized, as the specific surface area increases, the content of N-methyl-3-(N-methylamino)-propionamide (MMAP) in the solution increases, and in the case of the N,N′-dialkyl-3,3′-dithiodipropionamide according to the present invention, if the particle has a crystal size of less than 100 μm, remaining content of N-methyl-3-(N-methylamino)-propionamide (MMAP) may be increased a little, and if the solidified crystal size is greater than 2 mm, efficiency of a preparation process after solidification may not be increased.

To control the crystal size of N,N′-dialkyl-3,3′-dithiodipropionamide within the above-explained preferable range, the crystallization condition may be controlled in the solidification step. The solidification may be progressed at 0 to 30° C. for 1 to 3 hours. Meanwhile, to minimize the content of N-methyl-3-(N-methylamino)-propionamide (MMAP) in the product, the solidification may be preferably progressed at 0 to 20° C., more preferably 0 to 10° C., most preferably 0 to 5° C.

Under the above conditions, solidification may be progressed with stirring the reaction solution, and if stirring is progressed, stirring speed may be 30 to 200 rpm, preferably 60 to 120 rpm.

Meanwhile, if crystal is grown at a temperature of greater than 30° C. in the solidification step, the dissolved amount may be increased and thus it is economically infeasible, and at a temperature of 0° C. or less, there is a concern of freezing. Thus, the solidification may be preferably progressed at 0 to 30° C., and more preferably 0 to 10° C.

Further, if the solidification time is less than 1 hour, the crystal shape may not be uniform, and if the solidification time is greater than 3 hours, the temperature of the reaction solution may be increased.

Furthermore, in the solidification of N,N′-dialkyl-3,3′-dithiodipropionamide, at least one inorganic salt selected from the group consisting of sodium sulfate, ammonium sulfate, sodium chloride, ammonium chloride, magnesium sulfate, and magnesium chloride may be further introduced.

The inorganic salt may increase ionic strength of the reaction solution, increase the reaction yield due to salting-out, and maintain the solubility of N,N′-dialkyl-3,3′-dithiodipropionamide in the reaction solution low, thus allowing more efficient progress of the solidification. The inorganic salts that may be preferably used to efficiently progress the solidification and increase reaction yield may include sodium sulfate, ammonium sulfate, sodium chloride, magnesium chloride, and a combination thereof.

In order to decrease the solubility of N,N′-dialkyl-3,3′-dithiodipropionamide and increase efficiency of the solidification, the inorganic salt may be introduced in the content of 1 to 10 parts by weight based on 100 parts by weight of the reaction solution. If the introduced content is within the above range, the solubility of N,N′-dialkyl-3,3′-dithiodipropionamide in the reaction solution may be maintained in the preferable range of 5% or less.

As explained, by decreasing the solubility of N,N′-dialkyl-3,3′-dithiodipropionamide to 5% or less through temperature control and addition of an inorganic salt in the solidification step, N,N′-dialkyl-3,3′-dithiodipropionamide with a crystal size of 100 μm or more may be obtained.

According to yet another embodiment, a method further including centrifugal filtration after the solidification step is provided.

By passing the centrifugal filtration, the solidified N,N′-dialkyl-3,3′-dithiodipropionamide may be easily separated from the reaction solution. The centrifugal filtration may be preferably progressed such that moisture content of the solidified N,N′-dialkyl-3,3′-dithiodipropionamide may become 20 wt % or less.

More preferably, the centrifugal filtration may be preferably progressed such that moisture content of the solidified N,N′-dialkyl-3,3′-dithiodipropionamide may become 10 wt % or less, and most preferably 5 wt % or less.

As used herein, the term “moisture content” refers to the weight occupied by the residual amount of the reaction solution, based on the weight of the solid content, and is measured by the following method unless otherwise defined.

Specifically, when a hydrous sample is irradiated by a temperature-controllable infrared lamp, weight decrease is measured due to water evaporation, and weight decrease rate to the weight of the initial sample is represented by wt %. According to one embodiment, the KETT 600 model is used as moisture determination balance.

10 g of the sample is spread on a dish of the device for measuring moisture content, infrared rays are irradiated under a 105° C. condition for 10 minutes, and moisture content is calculated based on the weight change before and after irradiation.

For reference, if the moisture content of the solidified N,N′-dialkyl-3,3′-dithiodipropionamide is greater than 20 wt %, the content of N-methyl-3-(N-methylamino)-propionamide (MMAP) in the N,N′-dialkyl-3,3′-dithiodipropionamide solid may proportionally increase, and in the subsequent drying step, particles may be dissolved and a non-uniform mass may be produced. If the moisture content is too low, filtration time may be delayed. Thus, it may be preferable to select moisture content within the range of 20 wt % or less.

### DETAILED EMBODIMENTS OF THE INVENTION

Hereinafter, the present invention will be explained in detail referring to specific examples. However, these examples are provided only for better understanding of the invention, and the scope of the invention is not limited thereto.

To examine reaction yield according to the kinds of polar solvents and the content of MMAP in DDDA after solidification and drying, Examples 1 to 10 were conducted as follows.

### Example 1

### Preparation of N,N′-dimethyl-3,3′-dithiodipropionamide (hereinafter referred to as “DDDA”) in an aqueous solution

Into a 4-necked 3 L flask equipped with an agitator, a thermometer, a gas dispersion tube, a nitrogen purging adapter, and a cooling jacket, 3,3′-dithioproionic acid methyl ester (hereinafter referred to as “DDD) (944 g, 4 mol) and 1000 g of water were introduced. After filling the flask with nitrogen, the reaction solution was cooled to 5° C. While maintaining the reaction temperature 10° C. or less, monomethylamine (99%, 378 g, 12 mol) was added through the gas dispersion tube over about 4 hours. After completing introduction of monomethylamine, the mixture was stirred for 20 hours, and the reaction was completed at a reaction temperature of less than 10° C. At this time, in order to remove excessive monomethylamine and formed methanol, the mixture was heated to 50° C., and then evaporated under a vacuum degree of 100 mmHg. To solidify the produced N,N′-dimethyl-3,3′-dithiodipropionamide (DDDA), the reaction solution was slowly cooled for 100 minutes to lower the temperature of the reaction solution to 3° C. And then, to filter the formed slurry, the slurry was dehydrated using laboratory centrifugal filter (r=0.4 m, rpm=1700). The weight of the obtained solid after dehydration was 940 g, which was subsequently dried to a moisture content of 0.1% while maintaining at 50° C. under a vacuum degree of 10 mmHg for 3 hours. The weight of the final solid was 893 g (yield 94.5%), and moisture content was 5%. As the result of quantitative analysis, the content of remaining N-methyl-3-(N-methylamino)-propionamide (MMAP) impurities in the dried N,N′-dimethyl-3,3′-dithiodipropionamide (DDDA) was 5 ppm.

### Example 2

### Preparation of N,N′-dimethyl-3,3′-dithiodipropionamide (DDDA) in an aqueous solution including sodium sulfite

Into a 4-necked 3 L flask equipped with an agitator, a thermometer, a gas dispersion tube, a nitrogen purging adapter, and a cooling jacket, 3,3′-dithioproionic acid methyl ester (944 g, 4 mol) and 1000 g of water were introduced. Sodium sulfite (Na₂SO₃ 30 g) was introduced, and then the mixture was agitated to dissolve it. After filling the flask with nitrogen, the reaction solution was cooled to 5° C. While maintaining the reaction temperature 10° C. or less, monomethylamine (99%, 378 g, 12 mol) was added through the gas dispersion tube over about 4 hours. After completing introduction of monomethylamine, the mixture was stirred for 20 hours, and stirred at 10° C. to complete the reaction. At this time, in order to remove excessive monomethylamine and formed methanol, the mixture was heated to 50° C., and then evaporated under a vacuum degree of 100 mmHg. To solidify the produced N,N′-dimethyl-3,3′-dithiodipropionamide (DDDA), the reaction solution was slowly cooled for 100 minutes to lower the temperature of the reaction solution to 3° C. Then, to filter the formed slurry, the slurry was dehydrated using a laboratory centrifugal filter (r=0.4 m, rpm=1700). The weight of the obtained solid after dehydration was 940 g, which was subsequently dried to a moisture content of 0.1 wt % while maintaining it at 60° C. under a vacuum degree of 10 mmHg for 1 hour. The weight of the final solid was 906 g (yield 96%), and the moisture content was 5%. As the result of quantitative analysis, the content of remaining N-methyl-3-(N-methylamino)-propionamide (MMAP) impurities in the dried N,N′-dimethyl-3,3′-dithiodipropionamide (DDDA) was 0 ppm.

### Example 3

N,N′-dimethyl-3,3′-dithiodipropionamide (DDDA) was synthesized under the same conditions as Example 1, except for using a vacuum filter to filter a N,N′-dimethyl-3,3′-dithiodipropionamide (DDDA) slurry formed in the final filtering and drying steps. As a result of analysis, the N,N′-dimethyl-3,3′-dithiodipropionamide (DDDA) yield was 94%, and the content of N-methyl-3-(N-methylamino)-propionamide (MMAP) impurity was 10 ppm.

### Example 4

N,N′-dimethyl-3,3′-dithiodipropionamide (DDDA) was synthesized under the same conditions as Example 1, except for maintaining the reaction temperature at 25° C. to 30° C. As the result of analysis, the N,N′-dimethyl-3,3′-dithiodipropionamide (DDDA) yield was 91%, and the content of N-methyl-3-(N-methylamino)-propionamide (MMAP) impurity was 30 ppm.

### Example 5

N,N′-dimethyl-3,3′-dithiodipropionamide (DDDA) was synthesized under the same conditions as Example 2, except for maintaining the reaction temperature at 25° C. to 30° C. As a result of analysis, the N,N′-dimethyl-3,3′-dithiodipropionamide (DDDA) yield was 92%, and the content of N-methyl-3-(N-methylamino)-propionamide (MMAP) impurity was 5 ppm.

### Example 6

The experiment was conducted under the same conditions as Example 1, except that a methanol solvent was used as a polar solvent and methanol distillation was not conducted. The N,N′-dimethyl-3,3′-dithiodipropionamide (DDDA) yield was 74%, and the content of N-methyl-3-(N-methylamino)-propionamide (MMAP) impurity was 600 ppm.

### Example 7

Into a 4-necked 3 L flask equipped with an agitator, a thermometer, a gas dispersion tube, an nitrogen purging adapter, and a cooling jacket, 3,3′-dithioproionic acid methyl ester (944 g, 4 mol) was introduced. As a reaction solvent, 1000 g of ethanol was introduced. While maintaining the reaction temperature at 10° C. or less, monomethylamine (99%, 378 g, 12 mol) was added through the gas dispersion tube over about 4 hours. After completing introduction of monomethylamine, the mixture was stirred for 20 hours, and stirred while maintaining the inside temperature 10° C. to complete the reaction. At this time, excessive monomethylamine and formed methanol were distilled and removed under vacuum. To solidify the produced DDDA, the reaction solution was slowly cooled for 100 minutes to lower the temperature of the reaction solution to 3° C. Then, to filter the formed slurry, the slurry was dehydrated using a laboratory centrifugal filter (r=0.4 m, rpm=1700). Subsequently, the dehydrated solid was completed dried using a vacuum oven drier, and then the final weight was measured. As the result of quantitative analysis, the content of the remaining MMAP impurity in the dried DDDA was 700 ppm.

### Example 8

The experiment was conducted by the same method as Example 7, except for using 1000 g of propanol as the reaction solvent.

### Example 9

The experiment was conducted by the same method as Example 7, except for using 1000 g of isopropanol as the reaction solvent.

### Example 10

The experiment was conducted by the same method as Example 7, except for using 1000 g of butanol as the reaction solvent.

TABLE 1

| | | | | | | Remaining N- |
|---|---|---|---|---|---|---|
| | | | | | N,N′- | methyl-3-(N- |
| | | | | | dimethyl-3,3′- | methylamino)- |
| | | | Reaction | | dithiodipropionamide | propionamide |
| | Reaction | Reducing | temperature | Filtration | (DDDA) yield | (MMAP) content |
| | solvent | agent | (° C.) | method | (%) | in DDDA (ppm) |
| Example 1 | Water | — | 10 | Centrifugal | 94.5 | 5 |
| | | | | filtration | | |
| Example 2 | Water | Na₂SO₃ | 10 | Centrifugal | 96 | 0 |
| | | | | filtration | | |
| Example 3 | Water | — | 10 | Vacuum | 94 | 10 |
| | | | | filtration | | |
| Example 4 | Water | — | 25~30 | Centrifugal | 91 | 30 |
| | | | | filtration | | |
| Example 5 | Water | Na₂SO₃ | 25~30 | Centrifugal | 92 | 5 |
| | | | | filtering | | |
| Example 6 | Methanol | — | 10 | Centrifugal | 74 | 600 |
| | | | | filtration | | |
| Example 7 | Ethanol | — | 10 | Centrifugal | 75 | 700 |
| | | | | filtration | | |
| Example 8 | Propanol | — | 10 | Centrifugal | 77 | 750 |
| | | | | filtration | | |
| Example 9 | Isopropanol | — | 10 | Centrifugal | 76 | 740 |
| | | | | filtration | | |
| Example | Butanol | — | 10 | Centrifugal | 79 | 800 |
| 10 | | | | filtration | | |

Meanwhile, to examine DDDA yield and remaining MMAP content in the product according to the content of the polar solvent introduced in the reaction, Examples 11 to 15 were conducted as follows, and the results are described in Table 2.

### Example 11

Into a 4-necked 8 L flask equipped with an agitator, a thermometer, a gas dispersion tube, a nitrogen purging adapter, and a cooling jacket, 3,3′-dithioproionic acid methyl ester (944 g, 4 mol) was introduced. As the reaction solvent, water was introduced in the amount of 150% (1420 g) of DDD weight. While maintaining the reaction temperature at 5° C. or less, monomethylamine (99%, 378 g, 12 mol) was added through the gas dispersion tube over about 4 hours. After completing introduction of monomethylamine, the mixture was stirred for 20 hours, and stirred while maintaining the inside temperature at 10° C. to complete the reaction. At this time, excessive monomethylamine and formed methanol were distilled and removed under vacuum. To solidify the produced DDDA, the mixture was slowly cooled for 100 minutes to lower the temperature of the reaction solution to 3° C. Then, to filter the formed solid, the solid was dehydrated using a laboratory centrifugal filter (r=0.4 m, rpm=1700). Subsequently, the dehydrated solid was completely dried using a vacuum oven drier, and then the final weight was measured. The content of remaining MMAP impurity in DDDA was quantitatively analyzed and the result is described in the following Table 2.

### Example 12

The experiment was conducted by the same method as Example 11, except for using water in the content of 200% of the DDD weight as the reaction solvent.

### Example 13

The experiment was conducted by the same method as Example 11, except for using water in the content of 400% of the DDD weight as the reaction solvent.

### Example 14

The experiment was conducted by the same method as Example 7, except for using water in the content of 600% of the DDD weight as the reaction solvent.

### Example 15

The experiment was conducted by the same method as Example 7, except for using water in the content of 650% of the DDD weight as the reaction solvent.

TABLE 2

| | Example | Example | Example | Example | Example | |
|---|---|---|---|---|---|---|
| | 11 | 12 | 13 | 14 | 15 | Example 2 |
| Reaction solvent | water | water | water | water | water | water |
| Reaction solvent | 150 | 200 | 400 | 600 | 650 | 106 |
| content | | | | | | |
| (parts by weight, | | | | | | |
| based on 100 parts | | | | | | |
| by weight of DDD) | | | | | | |
| DDDA yield (%) | 96 | 95 | 92 | 91 | 88 | 94.5 |
| Remaining MMAP | 5 | 2 | 1 | 0 | 1 | 5 |
| content in DDDA | | | | | | |
| (ppm) | | | | | | |

Meanwhile, to examine reaction yield and MMMP content in DDDA according to the kind of the reducing agent, Examples 16 to 19 were conducted as follows, and the results are described in Table 3.

### Example 16

Into a 4-necked 8 L flask equipped with an agitator, a thermometer, a gas dispersion tube, a nitrogen purging adapter, and a cooling jacket, 3,3′-dithioproionic acid methyl ester (944 g, 4 mol) was introduced. As the reaction solvent, 1000 g of water was introduced. 30 g of potassium sulfite (K₂SO₃) was introduced, and the mixture was stirred to dissolve it. Then, while maintaining the reaction temperature at 10° C. or less, monomethylamine (99%, 378 g 12 mol) was added through the gas dispersion tube over about 4 hours. After completing introduction of monomethylamine, the mixture was stirred for 20 hours, and stirred while maintaining the inside temperature at 10° C. to complete the reaction. At this time, excessive monomethylamine and formed methanol were distilled and removed under vacuum. To solidify the produced DDDA, the mixture was slowly cooled for 100 minutes to lower the temperature of the reaction solution to 3° C. Then, to filter the formed solid, the solid was dehydrated using a laboratory centrifugal filter (r=0.4 m, rpm=1700). Subsequently, the dehydrated solid was completely dried using a vacuum oven drier, and then the final weight was measured. The content of MMAP impurity remaining in DDDA was quantitatively analyzed and the result is described in the following Table 3.

### Example 17

The experiment was conducted by the same method as Example 16, except for using 30 g of sodium thiosulfite (Na₂S₂O₃) as an additive, and the result is described in Table 3.

### Example 18

The experiment was conducted by the same method as Example 16, except for using 30 g of potassium thiosulfite (K₂S₂O₃) as an additive, and the result is described in Table 3.

TABLE 3

| | Exam- | Exam- | Exam- | Exam- |
|---|---|---|---|---|
| | ple 16 | ple 17 | ple 18 | ple 2 |
| Kind of reducing agent | K₂SO₃ | Na₂S₂O₃ | K₂S₂O₃ | Na₂SO₃ |
| DDDA yield (%) | 96 | 95 | 92 | 96 |
| Remaining MMAP content | 5 | 5 | 4 | 0 |
| (ppm) in DDDA | | | | |

To examine reaction yield and the remaining MMAP content in the reaction solution after reaction of DDD and the alkylamine according to the reaction temperature, Examples 19 to 29 were conducted as follows, and the results are described in Table 4.

### Example 19

Into a 4-necked 8 L flask equipped with an agitator, a thermometer, a gas dispersion tube, a nitrogen purging adapter, and a cooling jacket, 3,3′-dithioproionic acid methyl ester (944 g, 4 mol) was introduced. As the reaction solvent, 1000 g of water was introduced. While maintaining the reaction temperature at 0° C., monomethylamine (99%, 378 g 12 mol) was added through the gas dispersion tube over about 4 hours. After completing introduction of monomethylamine, the mixture was stirred for 20 hours, and the reaction was completed. At this time, excessive monomethylamine and formed methanol were distilled and removed under vacuum.

After the reaction was completed, to measure remaining MMAP content in the reaction solution, a sample was uniformly taken, and then the content of MMAP formed in the reaction solution was quantified, and the result is described in the following Table 4.

### Examples 20 to 23

Experiments were conducted by the same method as Example 19, except for changing the reaction temperature as described in the following Table 4, and the results are described in the following Table 4.

TABLE 4

| | Exam- | Exam- | Exam- | Exam- | Exam- |
|---|---|---|---|---|---|
| | ple 19 | ple 20 | ple 21 | ple 22 | ple 23 |
| Reaction temperature | 0 | 5 | 10 | 15 | 20 |
| (° C.) | | | | | |
| DDDA yield (%) | 96 | 96 | 96 | 96 | 96 |
| MMAP content in the | 480 | 500 | 630 | 660 | 780 |
| reaction solution (ppm) | | | | | |

Meanwhile, to examine reaction yield and remaining MMAP content in DDDA according to temperature condition in the solidification step, Examples 24 to 28 were conducted as follows, and the results are described in Table 5.

### Example 24

Into a 4-necked 8 L flask equipped with an agitator, a thermometer, a gas dispersion tube, a nitrogen purging adapter, and a cooling jacket, 3,3′-dithioproionic acid methyl ester (944 g, 4 mol) was introduced. As the reaction solvent, 1000 g of water was introduced. 30 g of sodium sulfite (Na₂SO₃) was introduced, and the mixture was agitated to dissolve it. While maintaining the reaction temperature at 10° C., monomethylamine (99%, 378 g, 12 mol) was added through the gas dispersion tube over about 4 hours. After completing introduction of monomethylamine, the mixture was stirred for 20 hours, and stirred while maintaining the inside temperature at 10° C. to complete the reaction. At this time, excessive monomethylamine and formed methanol were distilled and removed under vacuum. To crystallize the produced DDDA, the temperature of the reaction solution was lowered to 0° C., and then it was stirred for 100 minutes. To filter the formed solid, the solid was dehydrated using a laboratory centrifugal filter (r=0.4 m, rpm=1700). Subsequently, the dehydrated solid was completely dried using a vacuum oven drier, and then the final weight was measured. The content of remaining MMAP impurity in the dried DDDA was quantitatively analyzed and the result is described in the following Table 5.

### Examples 25 to 28

Experiments were conducted by the same method as Example 24, except for changing the solidification deriving temperature as described in the following Table 5, and the results are described in the following Table 5.

TABLE 5

| | Exam- | Exam- | Exam- | Exam- | Exam- |
|---|---|---|---|---|---|
| | ple 24 | ple 25 | ple 26 | ple 27 | ple 28 |
| Solidification | 0 | 5 | 10 | 20 | 30 |
| temperature (° C.) | | | | | |
| DDDA yield (%) | 97 | 96 | 95 | 91 | 85 |
| Remaining MMAP | 0 | 0 | 5 | 8 | 12 |
| content in DDDA (ppm) | | | | | |

Meanwhile, to examine reaction yield and remaining MMAP content according to change in moisture content of the DDDA solid substance in the centrifugation step, Examples 29 to 33 were conducted, and the results are described in Table 6.

### Example 29

Into a 4-necked 8 L flask equipped with an agitator, a thermometer, a gas dispersion tube, a nitrogen purging adapter, and a cooling jacket, 3,3′-dithioproionic acid methyl ester (944 g, 4 mol) was introduced. As the reaction solvent, 1000 g of water was introduced. 30 g of sodium sulfite (Na2SO3) was introduced, and the mixture was agitated to dissolve it. While maintaining the reaction temperature at 10° C., monomethylamine (99%, 378 g, 12 mol) was added through the gas dispersion tube over about 4 hours. After completing introduction of monomethylamine, the mixture was stirred for 20 hours, and stirred while maintaining the inside temperature at 10° C. to complete the reaction. At this time, excessive monomethylamine and formed methanol were distilled and removed under vacuum. To solidify the produced DDDA, the temperature of the reaction solution was lowered to 3° C., and then it was stirred for 10 minutes. To filter the formed solid, the solid was dehydrated using a laboratory centrifugal filter (r=0.4 m, rpm=1700). During the dehydration, rotation speed and time were controlled to control moisture content, and rpm was controlled such that the moisture content became 1 wt %. Subsequently, the dehydrated solid was completely dried using a vacuum oven drier, and then the final weight was measured. The content of remaining MMAP impurity in the dried DDDA was quantitatively analyzed and the result is described in the following Table 6.

### Examples 30 to 33

Experiments were conducted by the same method as Example 29, except for changing moisture contents during the centrifugal filtration after solidification as described in the following Table 6, and the results are described in the following Table 6.

TABLE 6

| | | Example | Example | Example | Example | Example |
|---|---|---|---|---|---|---|
| | Example 2 | 29 | 30 | 31 | 32 | 33 |
| Moisture content (wt %) | 5 | 1 | 5 | 10 | 15 | 20 |
| DDDA yield | 96 | 96 | 95 | 93 | 90 | 91 |
| (%) | | | | | | |
| Remaining MMAP content | 0 | 0 | 1 | 13 | 15 | 20 |
| in DDDA | | | | | | |
| (ppm) | | | | | | |

Meanwhile, to examine reaction yield and MMAP content remaining in DDDA according to the kind of inorganic salt added for solubility control in the solidification step, Examples 34 to 39 were conducted as follows, and the results are described in Table 7.

### Example 34

Into a 4-necked 8 L flask equipped with an agitator, a thermometer, a gas dispersion tube, a nitrogen purging adapter, and a cooling jacket, 3,3′-dithioproionic acid methyl ester (944 g, 4 mol) was introduced. As the reaction solvent, 1000 g of water was introduced. While maintaining the reaction temperature at 10° C., monomethylamine (99%, 378 g, 12 mol) was added through the gas dispersion tube over about 4 hours. After completing introduction of monomethylamine, the mixture was stirred for 20 hours, and stirred while maintaining the inside temperature at 10° C. to complete the reaction. At this time, excessive monomethylamine and formed methanol were distilled and removed under vacuum. Sodium sulfate, which is added to control solubility of the solid during solidification of the produced DDDA, was added in the amount of 23 g such that it may become 1% of the weight of the reaction solution, and then dissolved. The temperature of the reaction solution was lowered to 3° C., and then the mixture was stirred for 10 minutes. To filter the formed solid, the solid was dehydrated using a laboratory centrifugal filter (r=0.4 m, rpm=1700). During the dehydration, rotation speed and time of the centrifuge were controlled to control moisture content, and rpm was controlled such that moisture content became 1%. Subsequently, the dehydrated solid was completely dried using a vacuum oven drier, and then the final weight was measured. The content of remaining MMAP impurity in the dried DDDA was quantitatively analyzed and the result is described in the following Table 7.

### Examples 35 to 39

Experiment was conducted by the same method as Example 34, except adding inorganic salts for solubility control in the solidification step as described in Table 7, and the results are described in the following Table 7.

TABLE 7

| | Example | Example | Example | Example | Example | Example |
|---|---|---|---|---|---|---|
| | 34 | 35 | 36 | 37 | 38 | 39 |
| Kind of inorganic | Sodium | Ammonium | Sodium | Ammonium | Magnesium | Magnesium |
| salt | sulfate | sulfate | chloride | chloride | sulfate | chloride |
| DDDA yield (%) | 96 | 96 | 96 | 96 | 96 | 96 |
| Remaining | 0 | 0 | 1 | 4 | 5 | 1 |
| MMAP content in | | | | | | |
| DDDA (ppm) | | | | | | |

Meanwhile, reactions were progressed in the presence of a common non-polar inorganic solvent by the method of comparative examples, and the results are described in Table 8.

### Comparative Example 1

N,N′-dimethyl-3,3′-dithiodipropionamide (DDDA) was obtained by the same method as Example 1, except for using toluene as the reaction solvent instead of water. The N,N′-dimethyl-3,3′-dithiodipropionamide (DDDA) yield was 90%, and the content of remaining N-methyl-3-(N-methylamino)-propionamide (MMAP) impurity in DDDA was 5000 ppm.

### Comparative Example 2

The experiment was conducted by the same method as Example 7, except for using hexane as the reaction solvent instead of ethanol.

TABLE 8

| | | | | | | Content of |
|---|---|---|---|---|---|---|
| | | | | | | remaining N- |
| | | | | | N,N′-dimethyl- | methyl-3-(N- |
| | | | | | 3,3′- | methylamino)- |
| | | | | | dithiodipropionamide | propionamide |
| | Kind of | | Reaction | | (DDDA) | (MMAP) |
| | reaction | Reducing | temperature | Filtration | yield | in DDDA |
| | solvent | agent | (° C.) | method | (%) | (ppm) |
| Comparative | Toluene | — | 10 | Centrifugal | 90 | 5000 |
| Example 1 | | | | filtration | | |
| Comparative | Hexane | — | 10 | Centrifugal | 89 | 4700 |
| Example2 | | | | filtration | | |

### Experimental Example

Reaction yields, DDDA yields, and MMAP contents according to the above examples and comparative examples were measured and calculated as follows.

1. Moisture Content

A hydrous sample was irradiated by a temperature-controllable infrared lamp, weight decrease due to moisture evaporation was measured, and weight decrease rate to the initial weight was expressed in wt %. Moisture content of N′-dialkyl-3,3′-dithiodipropionamide according to one embodiment was measured and calculated by the following method.

As a device for measuring moisture content, the Moisture Determination Balance (MODEL: KETT 600) was used.

First, a sample was spread on a balance dish of the moisture content measuring device and about 10 g was taken. Then, under conditions of an infrared irradiation temperature of 105° C. and irradiation time of 10 minutes, the sample was dried to remove moisture for 10 minutes. The weight decrease rate of the sample before and after drying was expressed in %, and moisture content wt % was recorded.

2. DDDA Yield and Concentration

DDDA yield was obtained by the following equation.

Specifically, the weight of 3,3′-dithiopropionic acid methyl ester (DDD) introduced as a starting material was divided by its molecular weight (molecular weight 238) to calculate mole number, the weight of produced DDDA (molecular weight 236) was divided by its molecular weight to obtain mole number, and they were substituted in the following equation to calculate DDDA yield.

Equation

Yield %=(weight of produced DDDA g/236)/weight of introduced DDD g/238)*100

Meanwhile, the concentration of DDA was measured by HPLC (high performance liquid chromatography). Specific measuring conditions are as follows.

Composition of developing solvent:methanol/water=40/60

Detector wavelength: 254 nm

Column: C18 reverse phase

Pretreatment of sample: Solid DDDA is dissolved in a developing solvent to dilute it by 1000 times.

Retention Time: 4.5 minutes

DDDA purity %=(area of DDDA peak in the sample)/(area of DDDA peak in the standard)*100 Standard DDDA has purity of 99.9% or more.

3. Method for Measuring MMAP Content

The content of remaining MMAP in DDDA and the content of remaining MMAP in the reaction solution were measured as follows.

1) MMAP Standard Preparation

The following MMAP (molecular weight 116) and methyl acrylate (molecular weight 86.09) were reacted with methylamine (molecular weight 31.06) to prepare a standard.

Identification CAS: 50836-82-3

Formula: C₅H₁₂N₂O

IUPAC name: N-methyl-3-(N-methyl amino)-propanamide

2) Analysis Instrument and Method

Instrument name: API 4000 LC/MS/MS System

NANOSPACE HPLC System

Analysis was conducted by common LC/MASS method.

Pretreatment of sample: Solid DDDA was 10-fold diluted in methanol.

Drawing of calibration curve: A calibration curve of concentration and peak area was drawn using standard MMAP.

MMAP content: Calculated by substituting in the standard MMAP calibration equation and multiplying MMAP content in the sample by dilution rate.

As shown in all the examples, production of impurity N-methyl-3-(N-methylamino)-propionamide (MMAP) is significantly inhibited compared to the comparative examples.

While this disclosure has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A method for preparing N,N′-dialkyl-3,3′-dithiopropionamide comprising:
reacting 3,3′-dithiopropionic acid alkyl ester of the following Chemical Formula 2 with an alkylamine of the following Chemical Formula 3 at a temperature of from 0 to 50° C. in the presence of a polar solvent;
solidifying N,N′-dialkyl-3,3′-dithiodipropionamide of the following Chemical Formula 1 included in the reaction solution; and
drying the solid material of N,N′-dialkyl-3,3′-dithiodipropionamide: 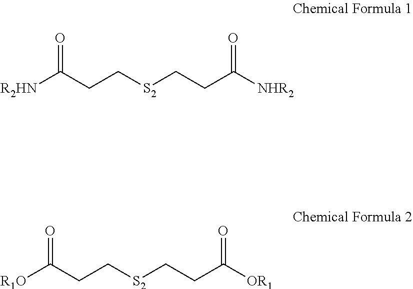
R₂NH₂ Chemical Formula 3
wherein, in Chemical Formulae 1 to 3, R₁ and R₂ are independently hydrogen or a C1-2 alkyl group.

2. The method according to claim 1, wherein the polar solvent is selected from an alcohol including a C1-10 linear or branched alkyl group, water, and a combination thereof.

3. The method according to claim 1, wherein the polar solvent is added in the content of 100 to 700 parts by weight, based on 100 parts by weight of the 3,3′-dithiopropionic acid alkyl ester of Chemical Formula 2.

4. The method according to claim 1, wherein the polar solvent is water.

5. The method according to claim 1, wherein the reaction is conducted while adding a reducing agent of an aqueous thiosulfite-based inorganic salt or a sulfite-based inorganic salt

6. The method according to claim 5, wherein the reducing agent is added in the equivalent of 0.1 to 1.0, based on 1 equivalent of the alkylamine.

7. The method according to claim 5, wherein the reducing agent is selected from Na₂S₂O₃, Na₂SO₃, K₂S₂O₃, K₂SO₃, and a combination thereof.

8. The method according to claim 1, wherein the solidification is progressed at 0 to 30° C. for 1 to 3 hours.

9. The method according to claim 1, wherein the solidification is progressed such that the crystal particle size of the N,N′-dialkyl-3,3′-dithiodipropionamide of Chemical Formula 1 may become 100 μm to 2 mm.

10. The method according to claim 1, wherein the solidification is progressed while adding at least one inorganic salt selected from the group consisting of sodium sulfate, ammonium sulfate, sodium chloride, ammonium chloride, magnesium sulfate, and magnesium chloride.

11. The method according to claim 10, wherein the inorganic salt is introduced in the content of 1 to 10 parts by weight, based on 100 parts by weight of the reaction solution supplied in the solidification step.

12. The method according to claim 1, further comprising centrifugal filtration, after the solidification step.

13. The method according to claim 12, wherein the centrifugal filtration is progressed such that moisture content of the solidified N,N′-dialkyl-3,3′-dithiodipropionamide may become 20 wt % or less.

14. The method according to claim 1, wherein the reaction temperature is 0 to 25° C.

15. The method according to claim 1, wherein the drying is progressed such that moisture content of the N,N′-dialkyl-3,3′-dithiodipropionamide is 0.1 wt % or less.
